(19) United States
(12) Patent Application Publication
(10) Pub. No.: US 2015/0110841 A1
(43) Pub. Date: Apr. 23, 2015

(54) USE OF POWDERED CELLULOSE IN COSMETIC APPLICATIONS

(71) Applicant: Evonik Industries AG, Essen (DE)

(72) Inventors: Susann Wiechers, Essen (DE); Frank Unger, Oberhausen (DE); Juergen Meyer, Essen (DE)

(73) Assignee: Evonik Industries AG, Essen (DE)

(21) Appl. No.: 14/382,449

(22) PCT Filed: Feb. 4, 2013
(86) PCT No.: PCT/EP2013/052125

§ 371 Date: Sep. 2, 2014

(30) Foreign Application Priority Data
Mar. 2, 2012 (DE) 10 2012 203 307.6

(51) Int. Cl. A61K 8/73; A61K 8/58; A61K 8/36; A61K 8/37; A61K 8/92; A61Q 1/06; A61K 8/81; A61Q 17/04; A61Q 19/00; A61Q 1/00; A61Q 1/10; A61K 8/06; A61K 8/34

(52) U.S. Cl. CPC A61K 8/731; A61K 8/062; A61K 8/585; A61K 8/36; A61K 8/37; A61K 8/375; A61K 8/92; A61K 8/345; A61K 8/8152; A61Q 17/04; A61Q 19/002; A61Q 1/00; A61Q 1/10; A61Q 1/06; A61K 2800/48; A61K 2800/52; A61K 2800/412; A61K 2800/592
USPC 424/401; 514/738

(57) ABSTRACT

The invention relates to a cosmetic formulation containing solid particles having a mean particle size of 3 μm to 20 μm, characterized in that the particles contain to at least 95% by wt. native cellulose, obtained from plant fibres, the percentages by weight relating to the dry total particle weight.

### FIELD OF THE INVENTION

The invention relates to a cosmetic formulation containing solid particles having a mean particle size of 3 μm to 20 μm, characterized in that the particles contain to at least 95% by wt. native cellulose obtained from plant fibres, the percentages by weight relating to the dry total particle weight.

### PRIOR ART

It is known that microcrystalline celluloses and cellulose derivatives are employed in cosmetics to stabilize cosmetic formulations.

EP1036799 describes the production of gel dispersions of unmodified and modified cellulose with a fraction of cellulose I-type crystal components of not more than 0.1 and a fraction of cellulose II-type crystal components of not more than 0.4. The aqueous sulphuric acid solution of the cellulose is diluted with water such that a gel-like cellulose suspension containing at most 6% of cellulose results. Dry cellulose particles or cellulose composite particles can be obtained by spray drying of the cellulose previously dissolved in aqueous sulphuric acid.

EP0264853 describes the production of covalently cross-linked cellulose particles, which are not larger than 300 μm. The particles are modified, e.g. to give cellulose xanthanate, in order to be able to carry out the cross-linking

EP1545433 discloses the production of cosmetically usable particles, the core of which contains a fluorescent component and additionally possesses a coating of cross-linked polyvinyl alcohol. Cellulose, inter alia, is listed as a core material modified in such a way. The particles should optically compensate skin imperfections by absorption of UV light and emission of visible light.

In WO2009085444, inter alia, spherical cellulose particles are used as macroparticles, in order to form a fractal network together with nanoparticles. This leads to the formation of gel structures in cosmetic formulations, which are used optically to reduce skin imperfections by the “Soft focus effect”.

EP1299069 describes the production of stable multiphase emulsions, in which particles are in the discontinuous phase. Modified celluloses are described as cellulose powders and mentioned as variants among many different fillers.

EP1372576 describes the use of modified celluloses and cellulose derivatives in self-foaming or foam-like cosmetic formulations.

In WO2009112375, emulsions are described in which the phase limits are stabilized by modified biopolymeric microparticles located in the phase interface. Covalently modified celluloses are described exclusively as suitable microparticles.

It was the object of the invention to make available a natural ingredient for cosmetic formulations which is able to reduce the tackiness of formulations, in particular containing moisture-donating polyols such as, for example, glycerol.

### DESCRIPTION OF THE INVENTION

Surprisingly, it has been found that native, non-derivatized, non-covalently modified cellulose particles solve the object set.

They produce a pleasant skin sensation.

The present invention therefore relates to cosmetic formulations containing solid particles as described in Claim 1.

A further subject of the invention is the use of such solid particles for the production of a cosmetic formulation.

An advantage of the present invention is that they produce a pleasant, non-tacky skin sensation.

Yet another advantage of the present invention is that the formulations are able to produce homogeneous and harmonic textures. Thus the cellulose particles according to the invention bring about a better spreadability of cosmetic formulations on the skin and an improved ability to be absorbed with simultaneously reduced slipperiness and tackiness.

Yet another advantage of the present invention is that the particles can bring about a stabilization of multiphase systems, such as, for example, of a cosmetic emulsion.

As a result of the administration form of the particles as dry particles with a high pure cellulose content, the entry of undesired by-products such as, for example, in cellulose dispersions or composite materials is prevented. By-products are undesired for health reasons, particularly in the use of ingredients in cosmetics, moreover, they can adversely affect the stability of the formulation.

Furthermore, it is advantageous that the native cellulose particles according to the invention were not changed by chemical modifications and also no chemically modified cellulose constituents were admixed to them. The particles according to the invention are thereby able to correspond very well to the wish of many consumers for cosmetic ingredients that are as natural as possible.

Yet another advantage of the present invention is that the particles do not or only very slightly influence the viscosity build of a cosmetic formulation.

Yet another advantage of the present invention is that the particles are able to bind high amounts of oil.

A further advantage of the present invention is that the particles have a homogeneous spreadability and more pleasant application behaviour, in particular in formulations based on polyacrylate-based thickeners.

The present invention relates to a cosmetic formulation that contains solid particles having a mean particle size of 3 μm to 20 μm, preferably of 3 μm to 15 μm, particularly preferably of 4 μm to 10 μm, which is characterized in that the particles contain to at least 95% by wt., preferably at least 97% by wt., particularly preferably at least 99% by wt., native cellulose obtained from plant fibres, the percentages by weight relating to the dry total particle weight.

Higher degrees of purity than 99.99% by wt. of native cellulose, obtained from plant fibres, based on the total particle weight are economically not justifiable.

The term “cosmetic formulation” in connection with the present invention is to be understood as meaning a composition which, in addition to the solid particles, contains at least one component, selected from water, cosmetic oils and cosmetic waxes. Preferably, the formulations according to the invention contain at least one further component selected from emollients, emulsifiers, thickeners/viscosity regulators/stabilizers, polyols and cosmetic active ingredients. Representatives of the individual groups are known to the person skilled in the art and can be taken, for example, from the German application DE 102008001788.4.

Particularly preferably, the formulations according to the invention are aqueous formulations, a water content of 5% by weight to 95% by weight, preferably 10% by weight to 90% by weight, particularly preferably 20% by weight to 80% by weight, based on the total formulation, being understood under the term “aqueous”.

In connection with the present invention, under the term “native cellulose obtained from plant fibres”, a cellulose is to be understood that has undergone no chemical modification in the form of an acid or base treatment, by which the amorphous fractions of the cellulose are at least partially removed, and in particular has undergone no chemical derivatization such as, for example, hydroxypropylation, hydroxyethylation, carboxymethylation, esterification (e.g. acetylation), etherification (e.g. methylation) and quaternization, but was obtained only from a natural substance by means of grinding in aqueous medium.

In connection with the present invention, under the term “dry”, a cellulose is described which has gone through the following drying:

10 g of the cellulose are stored at 105° C. in a drying cabinet for 2 hours.

The residual moisture before drying is at most 9%.

After cooling to room temperature in a desiccator, the sample is weighed out.

### Calculation:

100%−((weight after drying (g)×100%)/initial weight before drying (g))=(%) residual moisture

In connection with the present invention, under the term “solid”, the aggregate state “solid” is to be understood at an environmental temperature at which the cosmetic formulations are employed, this temperature range in particular extending from 15° C. to 45° C.

All conditions such as, for example, pressure and temperature, if not stated otherwise, are standard conditions (25° C., 1 bar). Percentages are indicated, if not described otherwise, in mass percent.

Laser scattering is employed in order to determine the mean particle size. For this purpose, the sample to be investigated is dispersed in deionized water with the aid of an ultrasonic bath (Bandelin SONOREX TK 30; dispersion time 5 minutes). The measurement is performed using the laser granulometer LS 230 from Beckman-Coulter. The material is measured using the SVM wet module, the calculation of the particle parameters takes place according to the Fraunhofer theory. The mean particle size is the d50 value in a volume-weighted particle size distribution.

The content of cellulose in the cellulose particles is determined as follows:

2.5 g of comminuted sample (determine dry content in a separate sample: 100%−TV % (TV: Loss on drying)) are weighed into a 150 ml beaker (E). By means of pipette, 30 ml of 17.5% sodium hydroxide solution temperature-controlled at 20° C. are added to the sample. The mass is carefully crushed using a glass rod, one end of which is flattened, and allowed to stand for 30 minutes. After this time, it is rapidly diluted with 100 ml of RO (reverse osmosis) water, immediately stirred and filtered off with suction. The glass frit G3 to be used is to be dried beforehand in a drying oven, and to be cooled in a desiccator and weighed (B_{empty}). It is to be rigorously ensured that the diluted mass is filtered off with suction from the alkaline liquid as rapidly as possible and also the subsequent washing is carried out rapidly. The washing out is to be carried out with RO water in portions. The mixture is washed until an alkaline reaction to pH paper no longer occurs. Thereupon, 0.5% strength hydrochloric acid is poured over the filter cake and it is allowed to stand for 10 minutes without filtering off with suction. It is then washed again with RO water until the pH paper no longer shows any acid reaction. The operation takes place at 20° C. The frit is then dried to constant weight at 105° C., cooled in a desiccator and weighed (B_{wt}).

$$Cellulose content ( \% ) = \frac{( {B}_{wt} - {B}_{empty} ) \times 100 \times 100}{E \times ( 100 - TV )}$$

A fundamental difference from the microcrystalline celluloses conventionally used as thickeners in cosmetics to the cellulose employed here is the characteristic that the particles are poorly water-soluble and act as solid particles in the cosmetic formulation. Therefore according to the invention cosmetic formulations are in particular preferred, which are characterized in that the particles have a maximal solubility in water at pH 7.0, 20° C., 1 bar, of 0 g/L to 0.5 g/L, preferably of 0 g/L to 0.2 g/L, particularly preferably of 0 g/L to 0.08 g/L.

The particles employed in the formulations according to the invention preferably contain a cellulose of a degree of crystallinity of 40 to 90%, preferably of 50 to 85%, particularly preferably of 60 to 80%.

For the quantitative determination of the crystallinity of cellulose samples, the following “peak height” method is used, described, for example, in N. Terinte, R. Ibbett and K. C. Schuster, Lenzinger Berichte 89 (2011) 118-131:

X-ray diffraction shots in the range from 5°-45° (2θ) are prepared in reflectance.

The air scattering curve is determined by means of the pure crystalline standard NIST640c, and this is used as a background for the X-ray diffraction diagrams of the measurement samples. This background is subtracted from the measurement sample. The degree of crystallinity CI is calculated as the ratio of the peak height of the crystalline signal I(002) at 22° (2θ) after the subtraction of the non-crystalline contribution I(non-crystalline) (the signal at 18° (2θ) and the peak height of the crystalline peak I(002) at 22° (2θ):

CI=((I(002)−I(non-crystalline))/I(002))*100%

According to the invention, cosmetic formulations are preferred which are characterized in that the crystalline portion of the cellulose contained in the particles does not correspond for the most part to cellulose type II. In particular, the content of cellulose type I in the crystalline portion is preferably greater than 95% by wt., particularly preferably greater than 99% by wt. based on the total crystallinity. The different types of cellulose are described, for example, in Park et al. Biotechnology for Biofuels 2010, 3:10. The determination of the cellulose type was carried out on the basis of a matching of the X-ray diffraction diagrams with the reference diagrams present in the ICSD database (Inorganic Crystal Structure Database). This matching took place on the basis of the peak positions and intensity ratios with the aid of the software search function of HighScore Plus (manufacturer: PANalytical), version: 3.0c.

According to the invention, cosmetic formulations are preferred, which are characterized in that the cellulose contained in the particles has a mean degree of polymerization of 1 to 50000, preferably of 50 to 20000, particularly preferably of 200 to 3000.

The average degree of polymerization is determined as follows by means of the measurement of the relative viscosity of the cellulose dissolved in a Cuen (copper(II)ethylenediamine) solution.

Initial weight of 1.3 g of sample in a 100 ml Erlenmeyer flask (WS). The dry content of the sample (DC) or the residual moisture is to be determined separately.

Final rinsing with 25 ml of RO water, which is flushed with nitrogen before use, and dispersion of the cellulose in the water by swirling.

Addition of 25 ml of 1M Cuen solution, which is flushed with nitrogen before use. Introduction of nitrogen into the sample solution. Closing of the Erlenmeyer flask with the glass stopper belonging to it. Shaking of the sample until the cellulose has completely dissolved. Transfer of about 15 ml of this solution to an Ubbelohde viscometer containing the 1c capillary. Temperature-controlling of the sample solution at 25° C.±0.1° C. Using a pipetting aid, the solution is drawn through the viscometer capillary until above the upper glass ball. Recording of the time which the solution needs to flow from the upper to the lower mark. Repetition of the process and calculation from both time measurements of the average value t1, if both values differ by not more than 1% from one another. Otherwise, a further determination is to be carried out and the average value of two results differing by not more than 1% from one another is to be determined. Repetition of the process without cellulose for the determination of the blank value. For this, the capillary 1 is used. The average value t2 obtained is the flow time of the pure Cuen solution.

### Calculation of the Relative Viscosity:

ηrel=(t1*k1)/(t2*k2)

t₁=flow time of the sample solution (average value)
t₂=flow time of the Cuen solution (average value)
k₁=constant of the Ubbelohde viscometer, capillary 1c
k₂=constant of the Ubbelohde viscometer, capillary 1

From the table 0315.-1. in Ph. Eur. 6.3. Powdered Cellulose, the value for the intrinsic viscosity to the relative viscosity for the solution of cellulose is inferred. The degree of polymerization is calculated as

PG=(9500*ηc)/E*(100−TV)

where
ηc: intrinsic viscosity, E: initial weight, TV: loss on drying is in %.

According to the invention, cosmetic formulations are preferred which are characterized in that the solid particles contained have a bulk density of 100-300 g/L, preferably 120-270 g/L, particularly preferably 140-240 g/L.

The bulk density is determined according to DIN 53468.

It is preferred according to the invention if the cosmetic formulation contains, based on the total formulation, 0.01% by wt. to 30% by wt., preferably 0.05% by wt. to 20% by wt., particularly preferably 0.1% by wt. to 10% by wt. of particles.

As the particles can be employed advantageously in multiphase systems, preferred formulations according to the invention are flowable formulations, in particular emulsions, in particular 0/W or W/O emulsions, having a viscosity of 0.01 Pas to 100000 Pas, preferably of 1 Pas to 20000 Pas, particularly preferably of 10 Pas, in particular 25 Pas, to 10000 Pas, this viscosity being measured at a shear rate of 10 s⁻1 and at 20° C.

Particularly good results have been achieved in cosmetic formulation if the particles have an oil absorption power of 1.0 g to 2.5 g, preferably of 1.2 g to 2.2 g, particularly preferably of 1.5 g to 2.0 g of cyclopentasiloxane per g of dry particles.

In an alternative embodiment of the present invention, similarly particularly good results have been achieved in cosmetic formulation if the particles have an oil absorption power of 1.0 g to 2.0 g, preferably of 1.2 g to 1.8 g, particularly preferably of 1.5 g to 1.7 g of diethyhexyl carbonate per g of dry particles.

In an alternative embodiment of the present invention, similarly particularly good results have been achieved in cosmetic formulation if the particles have an oil absorption power of 1.0 g to 2.0 g, preferably of 1.1 g to 1.7 g, particularly preferably of 1.4 g to 1.6 g of isopropyl myristate per g of dry particles.

In an alternative embodiment of the present invention, similarly particularly good results have been achieved in cosmetic formulation if the particles have an oil absorption power of 1.0 g to 2.0 g, preferably of 1.2 g to 1.8 g, particularly preferably of 1.45 g to 1.7 g of caprylic/capric triglycerides per g of dry particles.

In an alternative embodiment of the present invention, similarly particularly good results have been achieved in cosmetic formulation if the particles have an oil absorption power of 1.0 g to 2.5 g, preferably of 1.2 g to 2.2 g, particularly preferably of 1.5 g to 2.0 g of mineral oil per g of dry particles.

According to the invention, particularly preferred cosmetic formulations contain particles having an oil absorption power for cyclopentasiloxanes, diethyhexyl carbonates, isopropyl myristates, caprylic/capric triglycerides and mineral oil with the abovementioned particularly preferred ranges.

It has likewise proven advantageous if the cosmetic formulations according to the invention contain particles which have a water absorption power of 1 g to 3 g, preferably of 1.5 g to 3.5 g, particularly preferably of 1.7 g to 2.3 g of water of pH 7 per g of dry particle.

Very particularly preferred are the cosmetic formulations according to the invention which contain particles having an oil absorption power for cyclopentasiloxanes, diethyhexyl carbonates, isopropyl myristates, caprylic/capric triglycerides and mineral oil with abovementioned particularly preferred ranges and a water absorption power of 1.7 g to 2.3 g of water of pH 7 per g of dry particle.

The oil/water absorption power is determined by the following process:

2 g of particles are weighed into a Petri dish. The respective liquid (oil or water) is filled into a dropper bottle and weighed. Dropwise addition of the liquid and mixing of the particles and the liquid with a spatula takes place until a wet gloss is to be seen on the particles and no further liquid can be absorbed. The amount of oil needed is determined by reweighing the dropper bottle.

The formulations according to the invention particularly preferably contain as an additional component at least one polyol, as the particles can advantageously counteract the negative skin sensation of this component. In particular, the polyols are selected from polyols having 2 to 15 carbon atoms containing at least two hydroxyl groups. Typical examples are: glycerol, diglycerol, alkylene glycols, such as, for example, ethylene glycol, diethylene glycol, propylene glycol, butylene glycol, pentylene glycol, hexylene glycol, octylene glycol, and polyethylene glycols having an average molecular weight of 100 to 1,000 Daltons, technical oligoglycerol mixtures having a degree of autocondensation of 1.5 to 10 such as, for example, technical diglycerol mixtures having a diglycerol content of 40 to 50% by weight, methylol compounds, such as, in particular, trimethylolethane, trimethylolpropane, trimethylolbutane, pentaerythritol and dipentaerythritol, lower alkyl glucosides, in particular those having 1 to 4 carbon atoms in the alkyl radical, such as, for example, methyl and butyl glucoside, sugar alcohols having 5 to 12 carbon atoms, such as, for example, sorbitol or mannitol, sugars having 5 to 12 carbon atoms, such as, for example, glucose or sucrose, amino sugars, such as, for example, glucamine. Very particularly preferably, the formulation according to the invention contains glycerol, in particular in an amount from 0.001% by wt. to 20% by wt. preferably 0.01% by wt. to 15% by wt., particularly preferably 0.1% by wt. to 10% by wt., based on the total formulation.

In an alternative embodiment according to the invention, particularly preferred formulations according to the invention contain as an additional component at least one thickener selected from the group consisting of the polyacrylate-based thickeners, as the particles can advantageously counteract the negative skin sensation of this component. In particular, the polyacrylate-based thickeners are selected from polymers based on at least one of the following monomers: acrylic acid and its salts, alkyl esters of acrylic acid (e.g. C2-C30), hydroxyethyl acrylate, acrylamide, 2-acrylamido-2-methylpropanesulphonic acid and its salts (AMPS). In particular, the polyacrylate-based thickeners here are products with the INCI names selected from carbomer, sodium carbomer, acrylate copolymer, acrylamide, acrylamide/sodium acryloyldimethyl taurate copolymer, sodium acrylate/sodium acryloyldimethyl taurate copolymer, acrylic acid/vinyl pyrrolidone or sodium acrylate/acryloyldimethyl taurate/dimethylacrylamide crosspolymer.

Very particularly preferably, the alternative embodiment according to the invention contains the polyacrylate-based thickeners in an amount from 0.01% by wt. to 20% by wt., preferably 0.05% by wt. to 15% by wt., particularly preferably 0.1% by wt. to 10% by wt. based on the total formulation.

The use of the particles mentioned in connection with the formulations according to the invention for the production of cosmetics makes a contribution to the solution of the abovementioned object. Thus likewise a subject of the present invention is the use of solid particles having an average particle size of 3 μm to 20 μm, preferably of 3 μm to 15 μm, particularly preferably of 4 μm to 10 μm, for the production of a cosmetic formulation, characterized in that the particles consist to at least 95% by wt., preferably at least 97% by wt., particularly preferably at least 99% by wt., of native cellulose, obtained from plants fibres, the percentages by weight relating to the dry total particle weight.

In this use according to the invention, particularly preferred solid particles are used which are previously described above as being preferably contained in the formulations according to the invention.

In a further use according to the invention, solid particles having an average particle size of 3 μm to 20 μm, preferably of 3 μm to 15 μm, particularly preferably of 4 μm to 10 μm, are used for the reduction of the sticky skin sensation of a cosmetic formulation, characterized in that the particles consist to at least 95% by wt., preferably at least 97% by wt., particularly preferably at least 99% by wt., of native cellulose, obtained from plant fibres, the percentages by weight relating to the dry total particle weight.

In this use according to the invention, particularly preferred solid particles are used, which are previously described above as being preferably contained in the formulations according to the invention.

In a further use according to the invention, solid particles having an average particle size of 3 μm to 20 μm, preferably of 3 μm to 15 μm, particularly preferably of 4 μm to 10 μm, are used for better dispersibility of a cosmetic formulation, characterized in that the particles consist to at least 95% by wt., preferably at least 97% by wt., particularly preferably at least 99% by wt., of native cellulose obtained from plant fibres, the percentages by weight relating to the dry total particle weight.

In this use according to the invention, particularly preferred solid particles are used, which are previously described above as being preferably contained in the formulations according to the invention.

The examples listed below illustrate the present invention by way of example, without any intention of restricting the invention, the scope of application of which is apparent from the entirety of the description and the claims, to the embodiments specified in the examples.

### EXAMPLES

The examples listed below illustrate the present invention by way of example, without any intention of restricting the invention, the scope of application of which is apparent from the entirety of the description and the claims, to the embodiments specified in the examples.

The skin sensation of the cosmetic formulations described in the following examples was determined by a “panel”. At least five people compared the sensory properties of the cosmetic formulations and of the respective comparison formulation without knowing the composition. The properties are listed which the majority of the people described as preferable.

The nomenclature on the subject “stability” used for the assessment of the emulsion comparison examples is based on the following requirements. If the stability is assessed as “good”, this means that such an emulsion is stable for at least one month at room temperature, −5° C. and 40° C. “Stable” means here that no oil or water separation at all occurs, that the appearance of the emulsion remains homogeneous and that no significant changes in viscosity, colour or odour occur in the emulsion.

### Example 1 and Comparison Example V1

### Oil-in-Water Sunscreen Spray with a High Sun Protection Factor

The formulations indicated in Table 1 were prepared and their stability, appearance and skin sensation were assessed.

TABLE 1
according to Example 1: Formulations and results from
Example 1 and comparison example V1, oil-in-water sunscreen
spray with a high sun protection factor.

| Example | 1 | V1 |
|---|---|---|
| TEGO ® alkanol S 20 P | 1.00% | 1.00% |
| (Evonik Industries AG) | | |
| (Steareth-20) | | |
| TEGOSOFT ® TN (Evonik | 6.00% | 6.00% |
| Industries AG) (C12-15 alkyl | | |
| benzoate) | | |
| TEGOSOFT ® TIS (Evonik | 1.50% | 1.50% |
| Industries AG) | | |
| (triisostearin) | | |
| REWOPAL ® PIB 1000 | 1.00% | 1.00% |
| (Evonik Industries AG) | | |
| (polyisobutene) | | |
| TEGO ® Sun T 805 | 1.50% | 1.50% |
| (Evonik Industries AG) | | |
| (titanium dioxide; | | |
| trimethoxycaprylylsilane) | | |
| Bis-ethylhexyloxyphenol | 5.00% | 5.00% |
| methoxyphenyl triazine | | |
| Butyl methoxydibenzoylmethane | 5.00% | 5.00% |
| Diethylhexyl butamido triazone | 2.00% | 2.00% |
| Ethylhexyl methoxycinnamate | 1.00% | 1.00% |
| Ethylhexyl salicylate | 5.00% | 5.00% |
| Homosalate | 6.00% | 6.00% |
| Octocrylene | 5.00% | 5.00% |
| TEGO ® carbomer 341 ER | 0.50% | 0.50% |
| (Evonik Industries AG) | | |
| (Acrylates/C10-30 alkyl acrylate | | |
| crosspolymer) | | |
| Tocopheryl acetate | 0.50% | 0.50% |
| Ultrafine cellulose Arbocel M8, | 1.00% | 0.00% |
| J. Rettenmaier und Söhne | | |
| GmbH + Co. KG | | |
| glycerol | 5.00% | 5.00% |
| water | to 100% | to 100% |
| Phenylbenzimidazole sulphonic | 10.00% | 10.00% |
| acid (20% in water) | | |
| EDTA | 0.10% | 0.10% |
| Ethanol | 5.00% | 5.00% |
| Tris(hydroxymethyl)aminomethane | 1.50% | 1.50% |
| (30% in water) | | |
| Phenoxyethanol; ethylhexyl- | q.a. | q.a. |
| glycerol | | |
| Stability | Good | Good |
| Appearance | Yellowish, | Yellowish, |
| | homogeneous | homogeneous |
| Skin sensation | Markedly reduced | Very tacky, oily, |
| | tackiness, lower | poor absorption. |
| | oiliness, good | |
| | absorption. | |

### Example 2 and Comparison Example V2

### Oil-in-Water after-Shave Cream

The formulations indicated in Table 2 were prepared and their stability, appearance and skin sensation were assessed.

TABLE 2
according to Example 2: Formulations and results of Example 2
and comparison example V2, oil-in-water after-shave cream.

| | Example | |
|---|---|---|
| | 2 | V2 |
| Axol ® C 62 (Evonik Industries AG) | 1.50% | 1.50% |
| (glyceryl stearate citrate) | | |
| TEGIN ® M (Evonik Industries AG) | 2.00% | 2.00% |
| (glyceryl stearate) | | |
| TEGO ® Alkanol 1618 (Evonik | 3.00% | 3.00% |
| Industries AG) (cetearyl alcohol) | | |
| TEGOSOFT ® CT (Evonik | 3.50% | 3.50% |
| Industries AG) (caprylic/capric | | |
| triglyceride) | | |
| TEGOSOFT ® CR (Evonik | 2.00% | 2.00% |
| Industries AG) (cetyl ricinoleate) | | |
| TEGOSOFT ® TIS (Evonik | 1.00% | 1.00% |
| Industries AG) (triisostearin) | | |
| TEGOSOFT ® MM (Evonik | 0.50% | 0.50% |
| Industries AG) (myristyl myristate) | | |
| Cyclopentasiloxane | 4.00% | 4.00% |
| Jojoba Oil | 2.00% | 2.00% |
| Tocopheryl acetate | 0.50% | 0.50% |
| Cellulose particles from Example 1 | 2.00% | 0.00% |
| Glycerol | 4.00% | 4.00% |
| Panthenol | 0.50% | 0.50% |
| Allantoin | 0.20% | 0.20% |
| Water | to 100% | to 100% |
| TEGO ® carbomer 134 | 0.30% | 0.30% |
| (Evonik Industries AG) (carbomer) | | |
| Tegosoft ® OS (Evonik | 1.20% | 1.20% |
| Industries AG) (ethylhexyl stearate) | | |
| Bisabolol | 0.50% | 0.50% |
| 10% strength aqueous sodium | 0.90% | 0.90% |
| hydroxide solution | | |
| Methylisothiazolinone, | q.a. | q.a. |
| methylparaben, ethylparaben | | |
| Stability | Good | Good |
| Appearance | White, | White, |
| | homogeneous | homogeneous |
| Skin sensation | Easily | Poorly |
| | spreadable, | spreadable, |
| | quick | slow |
| | absorption, | absorption, |
| | little residue. | fatty residue |

### Example 3 and Comparison Example V3

### Natural Oil-in-Water Natural Cream

The formulations indicated in Table 3 were prepared and their stability, appearance and skin sensation were assessed.

TABLE 3
according to Example 3: Formulations and results of Example 3
and Comparison Example V3, Natural oil-in-water cream.

| | Example | |
|---|---|---|
| | 3 | V3 |
| TEGO ® Care PSC3 (Evonik | 2.50% | 2.50% |
| Industries AG) (polyglyceryl-3 | | |
| dicitrate/stearate) | | |
| TEGIN ® M (Evonik | 1.20% | 1.20% |
| Industries AG) (glyceryl stearate) | | |
| TEGO ® Alkanol 18 (Evonik | 1.30% | 1.30% |
| Industries AG) (stearyl alkohol) | | |
| TEGOSOFT ® P (Evonik | 6.50% | 6.50% |
| Industries AG) (isopropyl palmitate) | | |
| TEGOSOFT ® TIS (Evonik | 3.50% | 3.50% |
| Industries AG) (triisostearin) | | |
| Almond oil (Prunus dulcis) | 6.00% | 6.00% |
| Cellulose particles from Example 1 | 1.00% | 0.00% |
| Glycerol | 10.00% | 10.00% |
| Water | to 100.00% | to 100% |
| 10% strength aqueous sodium | 0.20% | 0.20% |
| hydroxide solution | | |
| Rokonsal BSB-N (ISP) (benzyl | 0.80% | 0.80% |
| alcohol, glycerol, benzoic acid, | | |
| sorbic acid) | | |
| Stability | Good | Good |
| Appearance | White, | White, |
| | homogeneous | homogeneous |
| Skin sensation | Reduced | Tacky, |
| | tackiness, | oily/moist |
| | lower oiliness, | residue, poor |
| | good absorption, | absorption. |
| | little residue. | |

### Example 4 and Comparison Examples V4.1, V4.2 and V4.3 and V4.4

### Emulsifier-Free Oil-in-Water Emulsion

The formulations indicated in Table 3 containing cellulose particles from Example 1, without cellulose and with three types of microcrystalline cellulose were prepared and their viscosity, stability, appearance and the sensory properties were assessed on spreading on the skin.

TABLE 4
according to Example 4: Formulations and results of Example 4 (with cellulose
particles from Example 1) and Comparison Examples V4.1 (without cellulose particles),
V4.2, V4.3 and V4.4 (in each case with Microcrystalline Cellulose), emulsifier-free oil-
in-water emulsion.

Example

| | 4 | V4.1 |
|---|---|---|
| TEGOSOFT ® OP (Evonik Industries AG) | 4.00% | 4.00% |
| (ethylhexyl palmitate) | | |
| TEGOSOFT ® DEC (Evonik Industries | 4.00% | 4.00% |
| AG) (diethylhexyl carbonate) | | |
| TEGOSOFT ® CT (Evonik Industries AG) | 4.00% | 4.00% |
| (caprylic/capric triglyceride) | | |
| TEGOSOFT ® TN (Evonik Industries AG) | 3.00% | 3.00% |
| (C12-15 alkyl benzoate) | | |
| TEGOSOFT ® M (Evonik Industries AG) | 2.50% | 2.50% |
| (isopropyl myristate) | | |
| Tocopheryl Acetate | 0.50% | 0.50% |
| TEGO ® Carbomer 341 ER (Evonik | 0.30% | 0.30% |
| Industries AG) (acrylates/C10-30 alkyl | | |
| acrylate crosspolymer) | | |
| Cellulose particles from Example 1 | 1.00% | 0.00% |
| Glycerol | 3.00% | 3.00% |
| Water | to 100% | to 100% |
| Propylene glycol | 2.00% | 2.00% |
| Panthenol | 0.50% | 0.50% |
| 10% strength aqueous sodium hydroxide | 0.90% | 0.90% |
| solution | | |
| Phenoxyethanol; ethylhexylglycerol | q.a. | q.a. |
| Viscosity Brookfield DV-I Prime spindle 4 | 28 Pas | 28 Pas |
| at 5 rpm | | |
| Stability | Good | Good |
| Appearance | White, | White, |
| | homogeneous | homogeneous |
| Sensory properties on spreading on the | Easy to spread, | Easy to spread, |
| skin | hardly oily, good | inhomogeneous, |
| | absorption | water and oil |
| | | phases separate on |
| | | the skin. |

| | Example | | |
|---|---|---|---|
| | V4.2 | V4.3 | V4.4 |
| TEGOSOFT ® OP | 4.00% | 4.00% | 4.00% |
| (Evonik Industries AG) | | | |
| (ethylhexyl palmitate) | | | |
| TEGOSOFT ® DEC | 4.00% | 4.00% | 4.00% |
| (Evonik Industries AG) | | | |
| (diethylhexyl carbonate) | | | |
| TEGOSOFT ® CT | 4.00% | 4.00% | 4.00% |
| (Evonik Industries AG) | | | |
| (caprylic/capric | | | |
| triglyceride) | | | |
| TEGOSOFT ® TN (Evonik | 3.00% | 3.00% | 3.00% |
| Industries AG) (C12-15 | | | |
| alkyl benzoate) | | | |
| TEGOSOFT ® M (Evonik | 2.50% | 2.50% | 2.50% |
| Industries AG) (isopropyl | | | |
| myristate) | | | |
| Tocopheryl acetate | 0.50% | 0.50% | 0.50% |
| TEGO ® Carbomer 341 | 0.30% | 0.30% | 0.30% |
| ER (Evonik Industries | | | |
| AG) (acrylates/C10-30 | | | |
| alkyl acrylate | | | |
| crosspolymer) | | | |
| AVICEL CL-611 (FMC | 1.00% | 0.00% | 0.00% |
| BioPolymer) | | | |
| (microcrystalline | | | |
| cellulose) | | | |
| AVICEL PC-611 (FMC | 0.00% | 1.00% | 0.00% |
| BioPolymer) | | | |
| (microcrystalline | | | |
| cellulose, cellulose gum) | | | |
| AVICEL PH-101 (FMC | 0.00% | 0.00% | 1.00% |
| BioPolymer) | | | |
| (microcrystalline | | | |
| cellulose) | | | |
| Glycerol | 3.00% | 3.00% | 3.00% |
| Water | to 100% | to 100% | to 100% |
| Propylene glycol | 2.00% | 2.00% | 2.00% |
| Panthenol | 0.50% | 0.50% | 0.50% |
| 10% strength aqueous | 0.90% | 0.90% | 0.90% |
| sodium hydroxide | | | |
| solution | | | |
| Phenoxyethanol; | q.a. | q.a. | q.a. |
| ethylhexylglycerol | | | |
| Viscosity Brookfield DV-I | 34 Pas | 37 Pas | >40 Pas |
| Prime | | | (too viscous, not |
| Spindle 4 at 5 rpm | | | measurable) |
| Viscosity Brookfield DV-I | | | 20 Pas |
| Prime | | | |
| Spindle c at 10 rpm | | | |
| Stability | Good | Good | Good |
| Appearance | White, homogeneous | White, homogeneous | White, homogeneous |
| Sensory properties on | Poor to spread, | Poor to spread, | Greasy, |
| spreading on the skin | water and oil | greasy, | inhomogeneous, |
| | phases separate | inhomogeneous, water | water and oil phases |
| | on the skin. | and oil phases | separate on the skin. |
| | | separate on the skin. | |

### Further Formulation Examples:

### Natural Water-in-Oil Cream

| | |
|---|---|
| ISOLAN ® PDI (Evonik Industries AG) (diisostearoyl | 3.00% |
| polyglyceryl-3 dimer dilinoleate) | |
| Castor wax | 0.40% |
| Beeswax | 0.60% |
| TEGOSOFT ® CT (Evonik Industries AG) (caprylic/capric | 7.00% |
| triglyceride) | |
| TEGOSOFT ® OER (Evonik Industries AG) (oleyl erucate) | 4.00% |
| TEGOSOFT ® TIS (Evonik Industries AG) (triisostearin) | 2.00% |
| TEGOSOFT ® CR (Evonik Industries AG) (cetyl ricinoleate) | 1.00% |
| Avocado oil | 5.00% |
| Cellulose particles from Example 1 | 1.00% |
| Glycerol | 7.00% |
| Water | to 100% |
| Magnesium sulphate heptahydrate | 1.00% |
| Euxyl K 712 (Schülke & Meyer) | 0.50% |
| (Aqua, sodium benzoate, potassium sorbate) | |
| 10% strength aqueous citric acid | Adjust |
| | pH to 5 |

Oil-in-Water Lotion with pH=4

| | |
|---|---|
| ABIL ® Care XL 80 (Evonik Industries AG) | 2.50% |
| TEGOSOFT ® Liquid (Evonik Industries | 5.00% |
| AG) | |
| TEGOSOFT ® DC (Evonik Industries AG) | 5.00% |
| (decyl cocoate) | |
| TEGOSOFT ® M (Evonik Industries AG) | 5.00% |
| (isopropyl myristate) | |
| Sepigel 305 (SEPPIC) (polyacrylamide | 1.50% |
| and C13-14 isoparaffin and laureth-7) | |
| Cellulose particles from Example 1 | 1.00% |
| Water | to 100% |
| Propylene glycol | 3.00% |
| 10% strength aqueous citric acid | Adjust to pH = 4 |
| Methylisothiazolinone, methylparaben, | q.a. |
| ethylparaben | |

### Water-in-Oil Serum

| | |
|---|---|
| ABIL ® EM 90 (Evonik Industries AG) (Cetyl PEG/PPG- | 1.50% |
| 10/1 dimethicone) | |
| ABIL ® EM 97 S (Evonik Industries AG) (Bis-PEG/PPG- | 1.00% |
| 14/14 dimethicone•dimethicone) | |
| Cyclopentasiloxane | 12.00% |
| TEGOSOFT ® DEC (Evonik Industries AG) (diethylhexyl | 3.00% |
| carbonate) | |
| HyaCare ® Filler CL (Evonik Industries AG) (aqua; | 2.50% |
| ethylhexyl stearate; sodium hyaluronate | |
| crosspolymer; polyglyceryl-4 | |
| diisostearate/polyhydroxystearate/ | |
| Sebacate; sodium isostearate) | |
| Tocopherol | 0.50% |
| Zinc stearate | 0.50% |
| Water | to 100% |
| Glycerol | 4.00% |
| Butylene glycol | 4.00% |
| Sodium chloride | 0.80% |
| TEGO ® PEP 4-17 (Evonik Industries AG) (tetrapeptide- | 0.50% |
| 21; glycerol; butylene glycol; aqua) | |
| Cellulose particles from Example 1 | 1.00% |
| Phenoxyethanol; ethylhexylglycerol | q.a. |

### Oil-in-Water AP/Deo Lotion for Roll on Application

| | |
|---|---|
| TEGO ® Care PS (Evonik Industries AG) (methyl | 1.75% |
| glucose sesquistearate) | |
| TEGO ® Care PL 4 (Evonik Industries AG) | 0.25% |
| (polyglyceryl-4 laurate) | |
| TEGOSOFT ® DEC (Evonik Industries AG) | 3.00% |
| (diethylhexyl carbonate) | |
| TEGOSOFT ® PBE (Evonik Industries AG) (PPG-14 | 3.00% |
| butyl ether) | |
| TEGO ® Cosmo P 813 (Evonik Industries AG) | 0.50% |
| (polyglyceryl-3-caprylate) | |
| Cellulose particles from Example 1 | 1.00% |
| Water | to 100% |
| Natrosol 250HHR (Hercules) (hydroxyethylcellulose) | 1.00% |
| 50% strength aqueous aluminium chlorohydrate solution | 15.00% |
| Methylisothiazolinone, methylparaben, ethylparaben | q.a. |

### Oil-in-Water Men's Care Gel

| | |
|---|---|
| TEGOSOFT ® AC (Evonik Industries AG) (isoamyl | 2.00% |
| cocoate) | |
| TEGOSOFT ® OER (Evonik Industries AG) (oleyl | 2.00% |
| erucate) | |
| TEGOSOFT ® TIS (Evonik Industries AG) | 2.00% |
| (triisostearin) | |
| Cellulose particles from Example 1 | 1.00% |
| Water | to 100% |
| Glycerol | 5.00% |
| TEGO ® Carbomer 341 ER (Evonik Industries AG) | 0.50% |
| (Acrylates/C10-30 alkyl acrylate crosspolymer) | |
| HyaCare ® 50 (Evonik Industries AG) | 0.10% |
| (Hydrolysed hyaluronic acid) | |
| 10% strength aqueous sodium hydroxide solution | 1.50% |
| Methylisothiazolinone, methylparaben, | q.a. |
| ethylparaben | |

### Impregnating Solution for Wet Wipes

| | |
|---|---|
| TEGO ® Wipe Flex (Evonik Industries AG) | 5.70% |
| (Ethylhexyl stearate, phenoxyethanol, polyglyceryl-4- | |
| laurate, sorbitan laurate, dilauryl citrate) | |
| Cyclomethicone | 2.00% |
| Cellulose particles from Ex. 1 | 1.00% |
| Water | to 100% |
| Glycerol | 3.00% |
| TEGO ® Carbomer 141 (Evonik Industries AG) | 0.10%. |
| (Carbomer) | |
| Sodium hydroxide (10% in water) | q.s. |

### Make-Up Powder Foundation

| | |
|---|---|
| Zinc stearate | 3.00% |
| Mica | to 100% |
| Talc | 24.00% |
| Iron oxide | 5.00% |
| Cellulose particles from Example 1 | 10.00% |
| Titanium dioxide | 8.00% |
| Cetylethyl hexanoate | 2.00% |
| Squalane | 3.00% |
| Cetearylethyl hexanoate | 2.00%. |
| Mineral oil (30 mPas) | 2.00% |
| PEG/PPG-4/12 dimethicone | 1.00% |
| Aluminium starch octenylsuccinate | q.s. |
| Iron oxide | q.s. |
| Perfume | q.s. |

### Make-Up Foundation

| | |
|---|---|
| Phenyltrimethicone | 14.00% |
| Ethylhexyl palmitate | to 100% |
| Cetylethyl hexanoate | 5.00% |
| Carnauba wax | 4.70% |
| Stearoxydimethicone | 4.00% |
| PVP/eicosene copolymer | 1.00% |
| Cetylstearyl heptanoate | 2.85% |
| Covabead LH 85, polymethyl methacrylate particles | 3.00% |
| Silicon dioxide | 0.25%. |
| Zinc oxide | 7.00% |
| Nylon-1010 | 2.50% |
| Talc Covasil 4.05 | 9.50% |
| Acrylate copolymer | 2.00% |
| Cellulose particles from Example 1 | 2.50% |
| Aluminium starch octenylsuccinate | 9.50% |
| Iron oxide | 3.10% |
| Titanium dioxide (and) dimethicone | 14.50% |

### Eyeshadow Formulation

| Cyclopentasiloxane | to 100% |
|---|---|
| PPG-3 myristyl ether | 7.00% |
| ABIL ® WE 09 (Evonik Industries AG) | 1.00% |
| (Polyglyceryl-4-isostearate; cetyl PEG/PPG-10/1 | |
| dimethicone; hexyl laurate) | |
| Dimethicone (20 mPas) | 2.50% |
| Cera alba | 4.50% |
| Carnauba wax | 2.00% |
| A-C Copolymer 400 (ethylene/VA copolymer) | 2.50% |
| Ozocerite | 5.80% |
| C18-36-acid triglyceride | 2.00% |
| Liquipar oil (isobutylparaben (and) isopropylparaben (and) | 0.20% |
| butylparaben) | |
| Cellulose particles from Example 1 | 4.00% |
| Titanium dioxide | 5.00% |
| Chromium oxide (green) | 10.00% |
| CI 77491 (and) aluminium powder (and) silicon dioxide | 5.00% |
| CI 77891 (and) CI 77288 (and) mica | 10.00% |

Moisturizing Oil-in-Water Sunscreen Lotion with a High Sun Protection Factor

| | |
|---|---|
| TEGO ® Care PS (Evonik Industries AG) | 2.50% |
| (methyl glucose sesquistearate) | |
| TEGO ® Alkanol 1618 (Evonik Industries AG) | 0.75% |
| (cetearyl alcohol) | |
| TEGOSOFT ® XC (Evonik Industries AG) | 4.85% |
| (phenoxyethyl caprylate) | |
| TEGOSOFT ® DEC (Evonik Industries AG) | 2.00% |
| (diethylhexyl carbonate) | |
| Butyloctyl salicylate | 2.00% |
| Octocrylene | 2.00% |
| Butyl methoxydibenzoylmethane | 8.00% |
| Bis-ethylhexyloxyphenol methoxyphenyl triazine | 2.00% |
| Ethylhexyl salicylate | 3.00% |
| Ethylhexyl triazone | 2.00% |
| Glycerol | 4.00% |
| LACTIL ® (Evonik Industries AG) (sodium lactate; | 2.00% |
| sodium PCA; glycine; fructose; urea; niacinamide; inositol; | |
| sodium benzoate; lactic acid) | |
| Water | to 100% |
| Cellulose particles from Example 1 | 1.00% |
| TEGO ® Carbomer 341 ER (Evonik Industries AG) | 0.15% |
| (acrylates/C10-30 alkyl acrylate crosspolymer) | |
| Sodium hydroxide (10% strength in water) | 0.40% |
| Methylisothiazolinone, methylparaben; ethylparaben | q.a. |
| Perfume | q.a. |

### Lipstick Formulation

| | |
|---|---|
| Cyclopentasiloxane | 34.00% |
| Behenoxydimethicone | 3.00% |
| Stearyldimethicone | 10.00% |
| Polyisobutene | 5.00% |
| Phenyltrimethicone | 8.00% |
| Isododecane | 4.00% |
| Bis-diglyceryl polyacyladipate-2 | 4.00% |
| Ceresin | 24.00% |
| Titanium dioxide | 1.00% |
| Carmine red | 1.00% |
| D&C Red No. 7 | 3.00% |
| Polyethylene | 1.00% |
| Cellulose particles from Example 1 | 1.00% |
| Aluminium starch octenylsuccinate & lauroyllysine | 1.00% |

### Mascara Formulation

| | |
|---|---|
| Sucrose stearate | 4.00% |
| Polyglyceryl-3 methylglucose distearate | 2.00% |
| Stearyl alcohol | 1.00% |
| Candelilla wax | 5.00% |
| Carnauba wax | 1.75% |
| Beeswax | 4.25% |
| Hydrogenated rice bran wax | 5.00% |
| Adipic acid/diethylene glycol/glycerol crosspolymer | 5.00% |
| Ceramide NP | 0.05% |
| Iron oxide | 10.00% |
| Cellulose particles from Example 1 | 0.50% |
| Water | to 100% |
| 1,3-Butanediol | 3.00% |
| Triethanolamine | 1.80% |
| Acrylate/octylacrylamide copolymer | 5.00% |
| Phenoxyethanol; methylparaben; ethylparaben, butylparaben; | 0.60% |
| propylparaben, isobutylparaben | |

1. A cosmetic formulation comprising:
solid particles having a mean particle size of 3 μm to 20 μm, wherein said solid particles contain at least 95% by wt. native cellulose obtained from plant fibers, the percentages by weight relating to the dry total particle weight.

2. The cosmetic formulation according to claim 1, wherein said formulation contains, based on the total formulation, from 0.001% by wt. to 30% by wt. of said solid particles.

3. The cosmetic formulation according to claim 1, wherein said native cellulose has a degree of crystallinity of 40 to 90%.

4. The cosmetic formulation according to claim 1, wherein said solid particles have a maximal solubility in water at a pH of 7.0, a temperature of 20° C., and a pressure of 1 bar, of 0 g/l to 0.5 g/l.

5. The cosmetic formulation according to claim 1, wherein said native cellulose contained in said solid particles has a mean degree of polymerization of 1 to 50000.

6. The cosmetic formulation according to claim 1, wherein said solid particles have a bulk density of 100-300 g/L.

7. The cosmetic formulation according to claim 1, wherein said formulation is an emulsion having a viscosity at a shear rate of 10 s⁻1 and at a temperature of 20° C. of 0.01 Pas to 100000 Pas.

8. The cosmetic formulation according to claim 1, wherein said solid particles have an oil absorption power of 1.0 g to 2.5 g of cyclopentasiloxane per g of dry particles.

9. The cosmetic formulation according to claim 1, wherein said solid particles have an oil absorption power of 1.0 g to 2.0 g of diethyhexyl carbonate per g of dry particles.

10. The cosmetic formulation according to claim 1, wherein said solid particles have an oil absorption power of 1.0 g to 2.0 g of isopropyl myristate per g of dry particles.

11. The cosmetic formulation according to claim 1, wherein said solid particles have an oil absorption power of 1.0 g to 2.0 g of caprylic/capric triglycerides per g of dry particles.

12. The cosmetic formulation according to claim 1, wherein said solid particles have an oil absorption power of 1.0 g to 2.5 g of mineral oil per g of dry particles.

13. The cosmetic formulation according to claim 1, wherein said solid particles have a water absorption power of 1 g to 3 g of water of pH 7 per g of dry particles.

14. The cosmetic formulation according to claim 1, further comprising at least one polyol or at least one thickener selected from the group consisting of polyacrylate-based thickeners.

15. A method of forming a cosmetic formulation, said method comprising:
adding solid particles having an average particle size of 3 μm to 20 μm to at least one component selected from the group consisting of water, cosmetic oils and cosmetic waxes, wherein said solid particles consist to at least 95% by wt. of native cellulose obtained from plant fibers, the percentages by weight relating to the total particle weight.

16. (canceled)
